(19) United States
(12) Patent Application Publication
(10) Pub. No.: US 2024/0212809 A1
(43) Pub. Date: Jun. 27, 2024

(54) ORCHESTRATION OF MEDICAL REPORT MODULES AND IMAGE ANALYSIS ALGORITHMS

(71) Applicant: Smart Reporting GmbH, Munich (DE)

(72) Inventors: Wieland SOMMER, Munich (DE); Sigrid AUWETER, Puchheim (DE); Alexis LAUGERETTE, Munich (DE)

(21) Appl. No.: 17/913,786

(22) PCT Filed: Mar. 25, 2020
(86) PCT No.: PCT/EP2020/058426

§ 371 Date: Sep. 22, 2022

(51) Int. Cl. G16H 15/00; G06F 40/174; G06F 40/186

(52) U.S. Cl. CPC G16H 15/00; G06F 40/174; G06F 40/186

(57) ABSTRACT

Systems and methods for generating medical reports, includes analyzing interdisciplinary first medical data to automatically select report modules and to select, launch, and orchestrate image analysis algorithms. This is achieved through automated iterative selection of report modules and image analysis algorithms based on available interdisciplinary data including at least the results of the selected image analysis algorithms. This resulting information and data is characterized by a high level of detail and unambiguousness. Based on this information and data, a medical report is automatically created.

This invention pertains to the field of reporting on medical images and in particular of creating medical reports.

### BACKGROUND OF THE INVENTION

When generating a radiological report, the reviewing radiologist summarizes her or his observations made when reviewing medical images. In a first step, a typical radiology report is created organ by organ. E.g., in a CT of the thorax the radiologist is looking, beyond others, at the heart, pericardium, lung parenchyma and airways. In a subsequent step, the radiologist summarizes the main findings into an impression. The main findings of a report contain so-called key findings which indicate remarkable aspects found in the images.

The report can be free-text. Then, its structure, elements, style, wording and layout may differ from physician to physician. They are not machine-readable, not standardized, and not analyzable. Moreover, they are prone to artefacts and they might be unclear or even incomplete.

To overcome the draw backs of free-text reports, so-called structured reports were introduced. These are based on structured, machine-readable reporting templates that can be progressively filled in by radiologists. Ideally, a structured report is machine-readable, has a fixed structure and contains standardized elements, wording and layout. In addition, pre-generated report templates can be used. These may provide case-specific structure and include recommended reporting steps.

WO 2016/135100 A1 describes an approach to provide structured reports, which proposes the use of report modules as report building blocks based on decision trees. These are based on a hierarchical tree structure that also reflects dependencies of information. The resulting medical report is then created in a modular fashion, in that medical report modules can be added step-by-step during the creation of the medical report.

Typically, radiologists must perform several tasks in parallel when evaluating medical image data and preparing a report. First and foremost, they must analyze the medical images and summarize their observations and impressions in a radiological report. Nevertheless, they must further take into account additional information about the patient. This information can, for example, come from images of different modalities or measurement protocols, as well as from information in the patient's health record, laboratory findings, previous images, etc. Thus, the types and number of the individual reporting tasks to be undertaken depends on the available interdisciplinary data, i.e., medical images and other available patient specific medical data.

However, it often only becomes apparent during the process of reporting which data is actually used and which diagnostic steps have to be carried out. This is because new data and information are generated during this process, which may then indicate further steps. E.g., secondary findings may be made that require additional reporting tasks. Thus, the radiologist must always keep an overview of the initial and newly added data as well as the implications of the available data and information for their further procedure.

An approach to support the radiologist in maintaining an overview is made in WO 2016/125039 A1 by guiding the radiologist through the reporting process. There a communication system for dynamic checklists is described. The radiologist is provided with checklists when reporting images. Based on these checklists he or she is guided through the proposed reporting steps. The checklists are selected based on the patient data initially available and list items are automatically added or removed during the reporting process if necessary.

Furthermore, software tools are used frequently to support the analysis of medical images. Such tools can increase the precision of medical reports and the speed with which such reports are created. The radiologist may, e.g., use so-called computer-aided detection (CADe) or computer-aided diagnosis (CADx) algorithms. So far, in a typical scenario such algorithms are selected and used for image analysis in an isolated process and additional reporting tasks have to be started manually.

Thus, in addition to keeping an overview of the information and the reporting process a radiologist must also be familiar with the available software tools, select and start those tools that are suitable for his or her purposes, and finally incorporate their output data into his or her report and further procedure. In addition, algorithm results depend on the available data (e.g., a round lesion in the lung is more likely to be lung cancer rather than an unspecific lesion, if there is another lesion in the adrenal gland and suspicious lymph nodes). As described above, the available data may change during reporting.

An approach for automatic selection of a CADe algorithm is known from WO 2007/044504 A1. For each selected image or image volume, a suitable algorithm for the interpretation is selected based on image attributes, which can be obtained, e.g., from a DICOM header.

However, excessive use of software tools can occupy large amounts of memory and decrease computer system performance. Also, an unnecessarily large amount of additional information can make it difficult to keep track of relevant information. It is therefore important to determine a useful selection of available tools for each patient individually and to embed them efficiently in the reporting workflow.

An approach to automatically select and partially fill a suitable template for a medical report is described in US 2012/0035963 A1. Information is extracted from the patient's images and record and then used by a reasoning engine to make the selection. In addition, the information for findings detection and classification is fed into a CADx system, the results of which are intended to support radiologists in their findings.

Against this background, it is desirable to further improve the reporting workflow and computer system performance when creating a medical report. This improves the accuracy and completeness of medical reports, enhances the efficiency and speed of the reporting process and eventually results in better subsequent diagnosis of diseases and treatment of patients.

### SUMMARY OF THE INVENTION

The invention provides a method, a system and a computer program product for improving the workflow and computer system performance when creating a medical report.

According to one aspect of the invention a computer-aided method for generating medical reports is provided. The method comprises the following steps:
a. receiving first medical data of a patient comprising at least medical image data of the patient and additional available information related to the medical image data and/or the patient,
b. providing the first medical data as first input data,
c. selecting from a collection of report modules at least one report module based on the first input data,
d. selecting from a collection of image-analysis (IA) Algorithms at least one IA-algorithm for supporting the interpretation of the medical image data based on the first input data and/or the at least one type of the at least one selected report module,
e. supplying the selected at least one IA-Algorithm with the first input data as input parameters, and executing the at least one selected IA-Algorithm on a computer to produce output data,
f. filling in the at least one selected report module based at least on the output data of the at least one selected IA-Algorithm, wherein the filled in at least one selected report module contains second medical data,
g. providing the second medical data as second input data and add the second input data to the first input data, and
h. deciding, based on the first input data, whether
   i. steps c) to h) are carried out again, or
   ii. a medical report is created from the at least one report module selected and completed in the previous steps.

According to another aspect of the invention a system for generating medical reports is provided comprising
a receiving unit configured to receive first medical data of a patient comprising at least medical image data of the patient and additional available information related to the medical image data and/or the patient,
an input-output unit comprising a display device, a user input device and a processor configured to display medical reports and the received first medical data via the display device and to receive and process user inputs via the user input device, a medical report generating unit comprising a data storage component for storing a collection of report modules and a collection of IA-Algorithms, wherein the processor is programmed to
   a. provide the first medical data as first input data,
   b. select at least one report module from the collection of report modules based on the first input data,
   c. select at least one IA-algorithm for supporting the interpretation of the medical image data from the collection of IA-Algorithms based on the first input data and/or the at least one type of the selected at least one report module,
   d. supply the selected at least one IA-algorithm with the first input data as input parameters and execute the selected at least one IA-algorithm to produce output data,
   e. fill in the selected at least one report module based at least on the output data of the selected at least one IA-Algorithm, wherein the filled in selected at least one report module contains second medical data,
   f. provide the second medical data as second input data and add the second input data to the first input data,
   g. decide, based on the first input data, whether
      iii. steps b) to g) are carried out again, or
      iv. a medical report is created from the at least one report module selected and completed in the previous steps.

According to another aspect of the invention a computer program product stored on a non-transitory storage medium is provided comprising computer readable instructions to execute the steps of the of the above described method.

The foregoing and other advantages will appear from the following description. In the description, reference is made to the accompanying drawings which form a part hereof, and in which preferred embodiments of the invention are shown by way of illustration. Such embodiments do not necessarily represent the full scope of the invention, however, and reference is made therefore to the claims and herein for interpreting the scope of the invention.

### BRIEF DESCRIPTION OF THE DRAWINGS

FIG. 1 shows a flow chart illustrating an exemplary computer-aided method for generating medical reports:

FIG. 2 illustrates the transfer of data and information according to an exemplary embodiment of the invention;

FIG. 3 shows an exemplary system for generating medical reports according to the invention:

FIG. 4 shows a schematic user interface of an exemplary embodiment according to the invention:

FIG. 5 exemplifies the exemplary use of an exemplary embodiment according to the invention.

### DETAILED DESCRIPTION

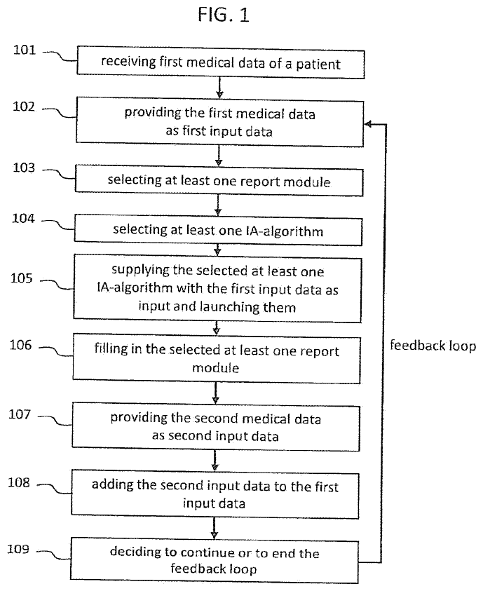

FIG. 1 illustrates the steps of a computer implemented method for generating medical reports according to an exemplary embodiment of the invention.

In step 101 first medical data is received. These data may either be sent by or retrieved from electronic data archiving systems, in particular databanks and repositories, like a picture archiving and communication system (PACS), a radiology information system (RIS), a hospital information system (HIS), and/or an electronic health record (EHR).

The received first medical data may consist of interdisciplinary data comprising all medical and other patient data that can influence decision making. This includes, but may not be limited to, medical images, clinical indications (e.g. “backpain”), tentative diagnoses (e.g. “suspected pancreatic carcinoma”), patient medical history, physical examination reports, exports from electronic health records (EHR), laboratory values, biopsies, medical reports and imaging examinations, image headers and other image parameters, and/or existing report templates. Interdisciplinary data can be derived from manual system entries, from direct interfaces to hospital information systems, from system outputs (feedback loop), and/or from automated analyses (e.g. automatically prioritized work lists). The medical images can, e.g., be acquired by imaging modalities like X-Ray, magnetic resonance imaging (MRI), computer tomography (CT), ultrasound (US), positron emission tomography (PET), single photon emission computed tomography (SPECT), digitalized microscopy images of tissues and/or other suitable imaging modalities. Combinations of medical images generated by different imaging modalities, e.g., overlays of PET- and MR-images, are further non-limiting examples for the medical images. The term medical images includes a single image as well as a set of several images, e.g., slices of a 2D or 3D multi-slice MR-acquisition or a set of images provided by different modalities mentioned above, for example, a prostate MRI scan and the corresponding digital pathology microscope-images.

In the following step 102, the first medical data are provided as first input data. In this step as well as in step 107, providing the medical data as input data may include preparing these data for further processing during the following steps. E.g., the information contained in the first and/or second medical data may be linked to a medical ontology. This may include an additional step of parsing the first and/or second medical data before linking them to an ontology.

Ontologies are sets of categories in the respective medical subject area that assign unambiguous meaning and define relations between categories. The ontologies used by the system may be private terms or codes, or standard codes such as RadLex or SNOMED CT, or a combination of several private and/or standard ontologies. Other medical data that is captured by the system can also be linked to these medical ontologies. Links between data elements and corresponding ontology terms may, e.g., be assigned manually, using natural language processing (NLP) assisted methods or other full-text search methods.

In step 103, one or more report modules are selected from a collection of report modules based on the first input data and in step 104 one or more image analysis (IA) algorithms are selected from a collection of IA-algorithms. Additionally, or alternatively, the user can manually select report modules and/or IA-algorithms, or change the automatic selections, e.g., by adding or removing report modules and/or IA-algorithms.

This automatic selection may be performed by a software instance, e.g., a decision support engine (DSE), which contains a collection of logic/algorithms that select report modules and IA-algorithms based on first medical data. In general, first medical data may influence IA-algorithms results (e.g., an automatically calculated risk score may depend on age or smoker status). To this end, the engine links the medical data to health knowledge contained within the system. The logic/algorithms contained in a DSE can be knowledge-based inference engines, e.g., based on clinical guidelines or published study results, or can be non-knowledge-based engines, e.g., based on machine learning. Furthermore, the logic/algorithms can be based on look-up tables. The DSE may in some embodiments be configured to access a large knowledge-base, for meaningful report module and IA-algorithm updates.

Report modules are report building blocks that consist of decision tree elements relating to a clinical reporting task. Such a modular approach makes the information contained in a report more accessible to dynamic updates. Report modules can be of varying depth, i.e., there can be any number of levels/nodes within the decision tree. An example of a report module would be a decision tree used to describe a lymph node lesion. If the DSE, or the user, concludes that a lymph node lesion might be present, this module will be selected to become part of the report. The module can then be filled manually by the user or it can be pre-filled by IA-algorithms. As explained above, report modules may also be rejected and/or deleted by the user. An example of one such module is the Lymph Node Module provided by Smart Reporting GmbH.

Report modules are hierarchical in nature, i.e., the individual elements are interdependent, and higher-order selections of elements determine the nature and possible values of lower level elements. This has two important consequences. First, automatic or manual selections and/or automated prefilling determine the possible next selections and/or entry options. These dependencies reduce complexity, because only such elements are provided that are currently needed instead of displaying all possible elements which may become very complex quickly. Due to this reduction in complexity, a very high granularity of information can be achieved, and memory usage and system performance can be improved. Second, report elements are embedded in a hierarchy, i.e., top level elements (e.g. “tumor”) group lower levels elements below them (e.g. “morphology”, “size”, “location”, etc.). This allows a high level of detail and assigning unambiguous meaning to individual report elements.

Hierarchical concepts are also used by medical ontologies (e.g., RadLex, which is also constructed as a tree) in order to unambiguously define terms and relationships between these terms. The hierarchical structure and interdependence of the elements of the report modules correspond essentially to the structure and dependencies of existing medical ontologies.

Due to these properties (hierarchy, high level of detail, unambiguousness), the individual elements of the report modules can be linked to elements/terms of medical ontologies and vice versa. Thus, by using medical ontologies the highly granular and unambiguous data/information can be stored and passed between the different components in an unambiguous machine-readable format.

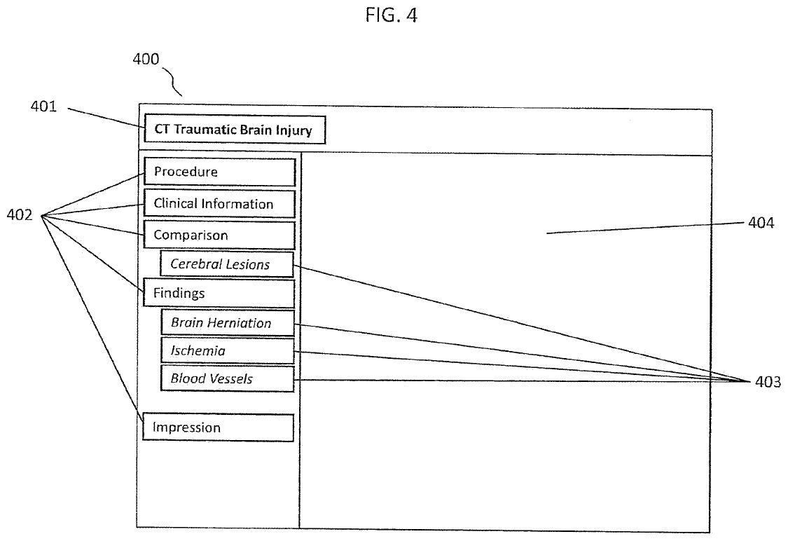

In some embodiments, the report modules may, e.g., be inserted into pre-existing report templates. In a separate step (not shown in FIG. 1), the DSE may, therefore, also select a suitable report template based on the first medical data. A report template is a full reporting-form that is used for a specific medical report. For radiology reporting, for example, there are typically five top-level elements: procedure, clinical information, comparison, findings, and impression (adapted MRRT standard). However, these elements may be modified or completely omitted. Each top-level element may consist of one or more report modules that are inserted depending on clinical need. In case there are no top-level elements, the template consists of a stack of modules. FIG. 4 shows a schematic illustration of user interface 400 with a loaded report template. This exemplary report template comprises the template name 401 (e.g. “CT Traumatic Brain Injury”), the top-level elements 402 (e.g. procedure, clinical information, comparison, findings, impression), the included report modules 403 (e.g. Cerebral Lesions, Brain Herniation, Ischemia and Blood Vessels, etc.) and a text field for synoptic report 404.

Continuing with FIG. 1, the selectable IA-algorithms are methods that assist in the interpretation of medical images. IA-systems process digital images, e.g., to detect lesions, segment tissue types and/or organs, determine parameters like diameters or volumes, classify findings (e.g. benign vs. malignant), calculate priorities or risk scores, detect pathologies, calculate pathology scores/probabilities, detect changes over time, automatically calculate distances or angles, visualize physiologic processes, preprocess or standardize images, differentiate between normal and abnormal findings, and/or analyze correct positioning of medical devices. Examples for classes of such IA-algorithm are known as computer aided detection (CADe) focusing on detection (e.g., location/segmentation of potential cancer), or computer aided diagnosis (CADx) algorithms focusing on diagnosis (e.g., classification “malignant” vs. “benign”). The output-data elements of IA-algorithms may be directly associated with report module elements, e.g., via a medical ontology and/or manually, and may be used to automatically pre-fill them. The selection of IA-algorithms can be independent of the selection of report modules. Alternatively, each report module may by default be associated with a set of IA-algorithms. In the latter case, the IA-algorithms are automatically loaded and may eventually be launched whenever the respective report module is selected, used, and/or inserted into a report template. A further approach combines report modules and sets of IA-algorithms as combined IA report-modules, that are defined by the individual combination of a report module with the set of IA-algorithms and that can be selected by the DSE in step 103 and 104 respectively.

In step 105, the first input data is then supplied to the IA-algorithms as input data/parameters. Depending on the specific implementation of the algorithms this may involve a further (not shown) step that prepares the necessary information, data and/or parameters contained in the first input data into a suitable input-format for the IA-algorithms. Depending on the specific IA-algorithm, only specific parts of the first input data may be used as input data, e.g., only the age, blood pressure and cholesterol levels of the patient as well as the information if she or he is a smoker or not. The IA-algorithms are then started automatically in step 105. Alternatively, the IA-algorithms may be launched manually by the user, e.g., the user launches the them individually or has to confirm their execution.

In step 106, the selected report module elements are then filled in based on the output data of the IA-algorithm/s. E.g., a finding of a new hyperintense periventricular lesion in a follow-up MRI scan of the brain by a lesion-detection algorithm would correspond to the specific nodes “Findings”/“T2 hyperintensity”/“Lesion”/“New”/“Periventricular” in the decision tree behind the report module “T2 hyperintensities”. This finding also corresponds to the specific nodes “Findings section” (RID28486)/“T2 hyperintensity” (RID39467)/“Lesion” (RID38780)/“New” (RID5720)/“Periventricular” (RID6384) in the decision tree behind the RadLex ontology. Therefore, the output data if the IA-algorithm, the filled-in report module elements and the ontology terms can be linked unambiguously.

Furthermore, the first medical data and/or the first input data can be used additionally to fill in the selected report module/s. In some embodiments, the user may fill in the selected report module/s or at least parts of it manually. The information on the patient that is contained in the filled in selected report modules represents additional medical data, also referred to as second medical data herein.

In step 107, the second medical data is then provided as second input data. As explained above, this can include preparing these data, e.g., by linking the data elements to a preexisting medical ontology (e.g. RadLex in the above example).

In step 108, the second input data is then added to the first input data, e.g., by concatenating the two data sets. This way, the amount of available information increases, which may indicate further reporting steps, which, in return, may require selecting and using further report modules and/or IA-algorithms. Alternatively, in some embodiments the second input data can replace the first input data. This leads to a smaller dataset saving memory and possibly increasing speed. However, it also reduces the amount of available information contained in the first input data.

In step 109, a decision is made whether steps 102-108 are repeated using the updated first input data or whether no more report modules and/or IA-algorithms are selected and launched, and the feedback loop is terminated by generating a structured report based on the filled in selected report modules. Possible criteria for the decision include the presence of new findings in the last iteration, reaching the end of reporting steps recommended by clinical guidelines and/or manual interaction by the user.

By repeating steps 102-108 a feedback loop is started. This way the report is iteratively adjusted based on interdisciplinary data. New data is generated with every cycle of the loop and based on this new information new report modules and IA-algorithms can be selected. Also, other data, e.g., existing images of other modalities or protocols, or even other organs may be included or, if these data is not yet available, its acquisition be recommended. Given that the granularity of information increases as the medical report progresses, the specificity of report modules and IA-algorithms usually increases. Consequently, less report modules and IA-algorithms have to be selected and used. This reduces complexity and increases the flexibility of the method improving the workflow and usability. Furthermore, this reduces the amount of data and active software applications to what is currently needed. Thereby, data handling, memory usage and system performance are optimized.

When deciding to terminate the feedback loop, the structured report is automatically generated based on the filled in report templates. Module entries are automatically transformed into a structured, standardized text that is displayed to the user and that can be modified, accepted, stored and exported into hospital information systems.

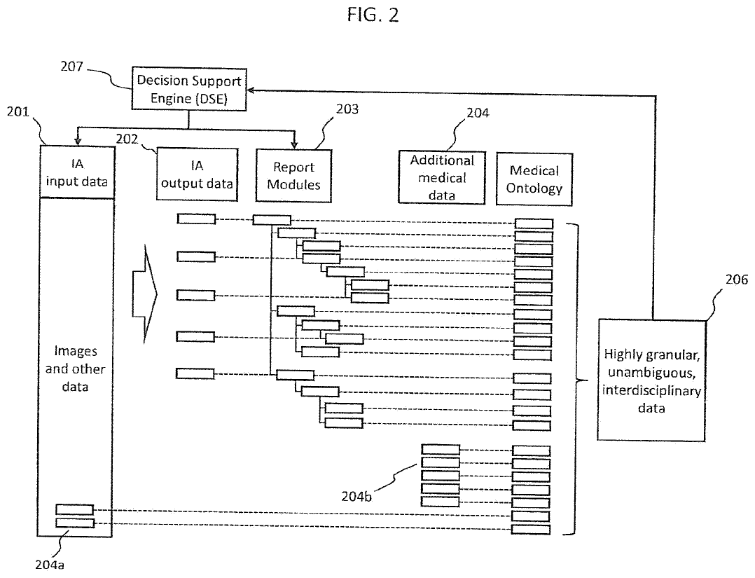

FIG. 2 illustrates the transfer of data and information according to an exemplary embodiment of the invention. The first input data is available as input data 201 of the IA-algorithm/s. These data consist of available medical image data and available additional non-image data of the patient.

The output data 202 of the selected IA-algorithms is then passed to the one or more report modules 203 and used to pre-fill their one or more entries/elements. According to one embodiment of the invention, a medical ontology is used to identify which output data corresponds to which entry of the report module. As discussed above, the elements of the ontology have unambiguous meaning. IA output-data as well as report-module elements can be linked to these ontology elements. Therefore, ontology elements can be used to establish an unambiguous correspondence between IA output-data and report-module elements. In other words, a medical ontology can serve as a common language making it possible to link different software and/or data components.

Furthermore, additional medical data 204 may be present that comprises first input data 204a that was not used to somehow fill in the currently edited report module/s and/or data 204b that was entered by the user in addition to or instead of the IA-outputs. Again, the additional medical data elements can be linked to elements of a medical ontology. In some embodiments (not shown), information and/or data that have the same meaning but come from different data sources may be linked to the same ontology element corresponding to that meaning. E.g., a finding “carcinoma” may result from radiological data as well of independent pathological data. Depending on the used ontology this may correspond to one or two ontological elements.

Thus, IA input data 201, IA output data 202, report module elements 203 and any additional medical data 204 are all linked to at least one common medical ontology 205 that assigns unambiguous meaning and relationships between terms. Thus, the resulting data 206 is fully machine readable, highly detailed, unambiguous and reflects relationships between terms. The machine readability and unambiguousness allow for the data to be used as input to the DSE 207. The high granularity of the data allows automation of medical workflows by meaningful iterative selection of report modules. The data furthermore serves as new input data for IA-algorithms and can, therefore, influence IA-algorithm results.

In conclusion, manual entries, IA outputs and additional information (e.g. from EHR) are captured and connected with a common, hierarchical ontology. These features allow the data to be used as input for a decision support engine that iteratively selects modules for the current patient/medical use case and provides this data to IA-algorithms as inputs. This, therefore, allows full dynamic orchestration of IA-algorithms and automation of medical reporting workflows.

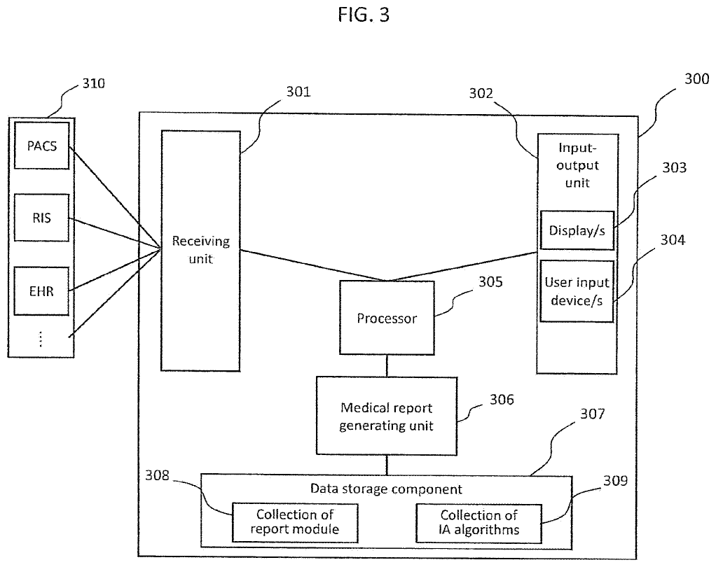

FIG. 3 schematically illustrates an exemplary system according to the invention.

A receiving unit 301 receives first medical data from an electronic data archiving system 310. The data can, e.g., be retrieved from or be sent by a PACS, RIS, HIS or EHR.

An input-output (IO) unit 302 comprises one or more display devices 303 and one or more user input devices 304. Additionally, the IO-unit may comprise further (not shown) output devices like speakers. Display devices include high-resolution displays configured for radiological workstations. User input devices may include, but are not limited to, a keyboard, a mouse, a trackpad, a touchscreen and/or a microphone. Furthermore, one or more processors 305 are part of the IO-unit and used to display medical reports and first medical data for the user, and to receive and process user inputs.

A medical report generating unit 306 that comprises a data storage component, e.g., a solid-state drive (SSD) a hard-disk drive (HDD) and also uses the processor 305. The processor 305 is programmed to execute steps 102-108 of the exemplary method described in FIG. 1.

Individual components of the system may be shared by several units, e.g., the processor 305 and/or the data storage component/s 307.

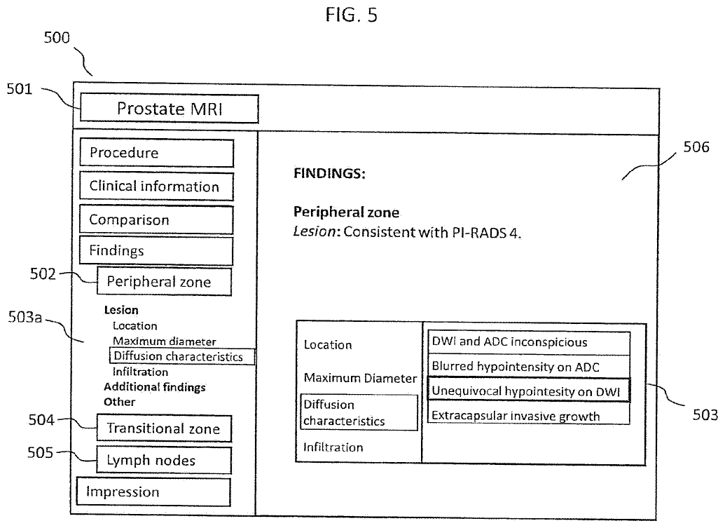

FIG. 5 exemplifies the functionality of an exemplary automated IA-algorithm and report module orchestration. In this example, a report template for prostate MRI is used as indicated by the respective field 501 of an exemplary user interface (UI) 500. The selected prostate segmentation and lesion detection-algorithm has identified a lesion in the peripheral zone of the prostate. This triggers insertion of the module “Peripheral Zone”, as indicated by the field 502 of the UI. The lesion's characteristics are captured using the layers and decision nodes of this module. Module layers may be displayed in pop-up windows 503 or side bars 503a or, as in this example, using a combination of both. Characteristics of the lesion (location, maximum diameter, etc.) are entered manually into the module or are automatically pre-filled using IA-algorithm outputs. In this example, the lesion characteristic “Diffusion characteristics” is currently edited and therefore, highlighted in the side bar and pop-up window of the UI. Entries are used to automatically generate a report text 506. Entries are also fed back into the decision support engine and are used to trigger insertion of additional report modules, E.g., based on the entries made into the report module “Peripheral Zone” above, the decision support engine triggered insertion of the report modules “Transitional Zone” and “Lymph Nodes” as indicated by the respective fields 504 and 505 of the UI. Report module entries may also influence further automated or manual filling in of report module entries, as entries can be interdependent.

The steps of the above described methods can be executed as computer readable instructions comprised in a computer program product. The computer program product can be stored on a storage medium. Examples for a storage medium include, but are not limited to, a hard drive, a CD or DVD or a Flash Memory. The computer program product can further be loaded, e.g., from an internet- or intranet-server or be electronically transmitted, e.g., via E-mail.

A general combined approach according to the invention is to analyze a vast array of interdisciplinary data in order to automatically select, launch and orchestrate IA-algorithms and to support medical reporting for subsequent diagnostic tasks. This is achieved through iterative, dynamic adjustment of report building blocks/modules and by influencing the results of one IA-algorithm based on other algorithmic results. The interdisciplinary information can then be used as input data for report modules and IA-algorithms. By additionally using complex report templates and report modules it is possible to generate information and data characterized by a high level of detail and unambiguousness. This way, it becomes possible to automatically generate a medical report, and orchestrate IA-algorithms.

In some embodiments, the structured report may be modified by the user, either by adjusting report modules and/or report module entries or by adjusting the report text directly. Once the user is satisfied with the report text, the report is finalized and the final report may be exported, e.g. to the user's hospital information system.

Besides being used for an application in reporting in radiology, embodiments of the invention can be used for a broad array of medical applications, including all diagnostic reporting tasks, as well as procedure documentation and referral letters. The methods, systems and computer programs according to the invention can also be used to generate interdisciplinary reports (e.g. combined radiology/pathology reports). In these interdisciplinary reports, each discipline can influence report module/IA-algorithm selection and IA-algorithm results in the other discipline(s).

The invention has been described with reference to the preferred embodiments. Modifications and alterations may occur to the skilled person upon reading and understanding the preceding detailed description. It is intended that the invention be construed as including all such modifications and alterations insofar as they come within the scope of the appended claims or the equivalents thereof.

What is claimed is:

1. A computer-implemented method for generating medical reports, the method including steps comprising:
a. receiving first medical data of a patient comprising at least medical image data of the patient and additional available information related to at least one of the medical image data or the patient,
b. providing the first medical data as first input data,
c. selecting from a collection of report modules at least one report module based on the first input data,
d. selecting from a collection of image-analysis (IA) Algorithms at least one IA-algorithm for supporting the interpretation of the medical image data based on at least one of the first input data or the at least one type of the at least one selected report module,
e. supplying the selected at least one IA-Algorithm with the first input data as input parameters, and executing the at least one selected IA-Algorithm on a computer to produce output data,
f. filling in the at least one selected report module based at least on the output data of the at least one selected IA-Algorithm, wherein the filled in at least one selected report module contains second medical data,
g. providing the second medical data as second input data and add the second input data to the first input data, and
h. deciding, based on the first input data, whether
i. steps c) to h) are carried out again, or
ii. a medical report is created from the at least one report module selected and completed in the previous steps.

2. The method according to claim 1, wherein the providing at least one of the first or second medical data includes linking the first or second medical data to a predetermined medical ontology.

3. The method according to claim 1, wherein selecting the at least one report module or IA-Algorithm is performed by a knowledge-based inference engine or a non-knowledge based inference engine, in particular based on machine learning.

4. The method according to claim 1, wherein the collection of report modules are constituted of decision tree elements each of which corresponds to a clinical reporting task.

5. The method according to claim 1, wherein the collection of IA-algorithms comprises at least one of computer-aided detection (CADe) algorithms or computer-aided diagnosis (CADx) algorithms.

6. The method according to claim 1, wherein filling in the selected at least one report module is based on at least one of the first input data or user input.

7. The method according to claim 1, wherein the deciding in step h) is further based on user input.

8. A system for generating medical reports, the system comprising:
a receiving unit configured to receive first medical data of a patient comprising at least medical image data of the patient and additional available information related to at least one of the medical image data or the patient,
an input-output unit comprising a display device, an user input device, and a processor configured to display medical reports and the received first medical data via the display device and to receive and process user inputs via the user input device,
a medical report generating unit comprising a data storage component for storing a collection of report modules and a collection of IA-Algorithms,
wherein the processor is programmed to;
a. provide the first medical data as first input data,
b. select at least one report module from the collection of report modules based on the first input data,
c. select at least one IA-algorithm for supporting the interpretation of the medical image data from the collection of IA-algorithms based on the first input data and/or the at least one type of the selected at least one report module,
d. supply the selected at least one IA-algorithm with the first input data as input parameters and execute the selected at least one IA-algorithm to produce output data,
e. fill in the selected at least one report module based at least on the output data of the selected at least one IA-algorithm, wherein the filled in selected at least one report module contains second medical data,
f. provide the second medical data as second input data and add the second input data to the first input data,
g. decide, based on the first input data, whether;
i. steps b) to g) are carried out again, or
ii. a medical report is created from the at least one report module selected and completed in the previous steps.

9. The system according to claim 8, wherein the processor is further programmed to provide the first or second medical data as first or second input data by linking the first or second medical data to a predetermined medical ontology.

10. The system according to claim 8, wherein the processor is further programmed to select the at least one report module or the at least one IA-algorithm via a knowledge-based inference engine or a non-knowledge based inference engine.

11. The system according to claim 8, in which report modules are constituted of decision tree elements each of which corresponds to a clinical reporting task.

12. The system according to claim 8, wherein the processor is further programmed to select at least one of computer-aided detection (CADe) algorithms or computer-aided diagnosis (CADx) algorithms as IA-algorithms.

13. The system according to claim 8, wherein the processor is further programmed to fill in the selected at least one report module based on at least one of the first input data or user input.

14. The system according to claim 1, wherein the processor is in step g) further programmed to decide based on user input.

15. A computer program product stored on a non-transitory storage medium comprising computer readable instructions to cause a processor to execute steps comprising:
a. receiving first medical data of a patient comprising at least medical image data of the patient and additional available information related to at least one of the medical image data or the patient,
b. providing the first medical data as first input data,
c. selecting from a collection of report modules at least one report module based on the first input data,
d. selecting from a collection of image-analysis (IA) Algorithms at least one IA-algorithm for supporting the interpretation of the medical image data based on at least one of the first input data or the at least one type of the at least one selected report module,
e. supplying the selected at least one IA-Algorithm with the first input data as input parameters, and executing the at least one selected IA-Algorithm on a computer to produce output data,
f. filling in the at least one selected report module based at least on the output data of the at least one selected IA-Algorithm, wherein the filled in at least one selected report module contains second medical data,
g. providing the second medical data as second input data and add the second input data to the first input data, and
h. deciding, based on the first input data, whether
i. steps c) to h) are carried out again, or
ii. a medical report is created from the at least one report module selected and completed in the previous steps.

16. The computer program product according to claim 15, wherein the providing at least one of the first or second medical data includes linking the first or second medical data to a predetermined medical ontology.

17. The computer program product according to claim 15, wherein selecting the at least one report module or IA-Algorithm is performed by a knowledge-based inference engine or a non-knowledge based inference engine.

18. The computer program product according to claim 15, wherein the collection of report modules are constituted of decision tree elements each of which corresponds to a clinical reporting task.

19. The computer program product according to claim 15, wherein the collection of IA-algorithms comprises at least one of computer-aided detection (CADe) algorithms or computer-aided diagnosis (CADx) algorithms.

20. The computer program product according to claim 15, wherein filling in the selected at least one report module is based on at least one of the first input data or user input.

21. The computer program product according to claim 15, wherein the deciding in step h) is further based on user input.
